(19) United States
(12) Patent Application Publication
(10) Pub. No.: US 2026/0130733 A1
(43) Pub. Date: May 14, 2026

(54) ROBOTIC SYSTEM AND CONTROL DYNAMICS FOR OCULAR SURGERY

(71) Applicant: Alcon Inc., Fribourg (CH)

(72) Inventor: Steven T. Charles, Memphis, TN (US)

(73) Assignee: Alcon Inc., Fribourg (CH)

(21) Appl. No.: 19/380,275

(22) Filed: Nov. 5, 2025

Related U.S. Application Data
(60) Provisional application No. 63/717,968, filed on Nov. 8, 2024.

(51) Int. Cl. A61B 34/00; A61B 34/32; A61B 34/35; A61F 9/007; B25J 9/16; B25J 13/08

(52) U.S. Cl. CPC A61B 34/77; A61B 34/32; A61B 34/35; A61F 9/00736; B25J 9/1633; B25J 9/1651; B25J 13/085

(57) ABSTRACT

A robotic system for performing an ocular surgery includes a robot and an electronic control unit (ECU). The robot includes an end-effector, six or more electric motors connected to or operable for moving six or more links and controllable via the ECU, and all-pose gravity compensation structure. A surgical tool is connected to the end-effector. The robot and surgical tool, in response to electronic control signals from the ECU, perform a surgical task on an eye during the ocular surgery. The ECU includes scaling logic operable for downscaling or limiting a tool force imparted to the eye by the surgical tool and/or a tool velocity of the surgical tool. All-pose gravity compensation minimizes a perceived weight of the surgical tool during the ocular surgery. The robotic system may include a surgeon-operated haptic device/master in which the robot acts as a slave device.

### CROSS-REFERENCE TO RELATED APPLICATIONS

This application claims the benefit of U.S. Provisional Patent Application No. 63/717,968, filed Nov. 8, 2024, which is incorporated by reference herein in its entirety, and is hereby expressly made a part of this specification.

### INTRODUCTION

Robots are used in a wide range of industrial and medical applications. For example, a surgical robot for use in performing microsurgery, including the various ocular surgeries noted herein, may include multiple links that are interconnected via revolute or prism joints to form an open or closed loop kinematic chain. A distalmost link connected to an end-effector is freely moveable within a defined operating space. Each respective joint of the robot is either actuated (“actively driven” or “active”) or unactuated (“passively moveable” or “passive”) to control joint angles and relative motion/orientation of the interconnected links. In addition to a surgical robot, a typical ophthalmic surgical suite utilizes a digital or analog microscope to enable the surgeon to view the patient's ocular anatomy under high degrees of magnification. The robot's motion through its available four to six degrees of freedom (DOF) ultimately enables the end-effector and surgical tools connected thereto to reach a desired position and orientation in free space, i.e., relative to the patient's eye.

Ocular surgery is typically performed by a surgeon while the surgeon views the patient's eye through the above-noted microscope or with the assistance of a digital visualization system and simultaneously manipulates one or more surgery-specific operating tools. At the same time, the surgeon controls the microscope's focus, zoom, position, and lighting via foot pedals or other interfaces, directs actions of support staff in the operating suite, and performs other relevant tasks. During a fully autonomous surgery, the surgical robot performs programmed surgical maneuvers in response to control signals from an electronic control unit or ECU. Alternatively, in a master-slave or tele-operated system, the surgeon provides input to a haptic device by manually controlling surgical maneuvers via a haptic interface, e.g., surgeon-graspable handles. The surgeon's manual inputs to the haptic interface are encoded for execution by the robot via the ECU. Such an arrangement is referred to herein and in the art as a master-slave system, i.e., one in which the haptic system acts as a master device and the robot acts as a slave device.

### SUMMARY

The present disclosure generally relates to robotic manipulation systems for performing a surgical procedure, and in particular to dexterity-enhanced manipulation systems for ocular microsurgical procedures. Aspects of the disclosure pertain to fully autonomous as well as master-slave implementations, and to two-limbed/bimanual as well as single-limbed surgeries. The above-summarized haptic interface may be located at a patient's bedside or at a remote console having stereo digital visualization capabilities. Robots whose motion is controlled herein may utilize arms/serial kinematics, hybrid parallel/serial kinematics, or parallel kinematics in different constructions.

Additionally, the present teachings envision pose-independent gravity compensation implemented in control law, e.g., an approach based on a smart connector disposed between the surgical tool(s) and the robot's end-effector that instructs an electronic control unit (ECU) regarding tool masses and moments. Pose-independent gravity compensation provides “weightless” tools and ensures that, in the unlikely event a surgeon should drop the haptic/master during surgery, the robot/slave/surgical tool does not fall down or move in response to the errant input. Pose-independent gravity compensation is intended to improve surgical safety and minimize surgeon fatigue. Disclosed features also include imaging sensors and, for the robot base, a passive counterbalanced structure, for instance a passive two-link/three vertical revolute joint mechanism using a counterweight or gas springs. Such a counterbalanced structure facilitates improved surgical safety, and in some cases, i.e., a counterbalanced robot frame attached to a patient's head, allowing the patient to move their head while the robot's pose remains constant relative to patient's head.

A robotic system in accordance with one or more representative embodiments may be used to perform an ocular surgery, for instance a vitreoretinal, cataract, cornea, or glaucoma surgery. An autonomous embodiment of the robotic system may be characterized by use of artificial intelligence (AI)-based control, stereo machine vision, polarization imaging, time-of-flight imaging and/or optical coherence tomography (OCT)-driven surgical operations. In an alternative master-slave embodiment, the surgeon may be situated at or near the patient's head, with the surgeon physically manipulating a six degree of freedom (6-DOF) haptic interface device. The various hardware and software solutions described below are intended to increase overall safety and precision relative to competing robot-assisted ocular surgeries.

The present robotic system in accordance with a representative/non-limiting embodiment includes an ECU and a surgical robot in communication therewith. The robot includes an end-effector, six or more links, and six or more electric motors connected to the link(s) and controllable via the ECU. A surgical tool such as forceps, a cutter, an irrigation/aspiration cannula, or a phacoemulsification tool is securely supported by the end-effector, for instance connected to the end-effector via a smart connector configured as set forth herein. In some implementations, a single end-effector supports interchangeable surgical tools attached to the end-effector via the above-noted smart connector. The surgeon's manual inputs to the haptic interface result in controlled movement of the robot(s), the end-effector(s), and ultimately the surgical tool(s).

The robot and surgical tool(s) are configured to perform a surgical task on a patient's eye during the ocular surgery. This action occurs in response to electronic control signals from the ECU. The ECU is programmed with scaling logic operable for downscaling or limiting a tool force imparted to the eye by each surgical tool, and for downscaling or limiting a tool velocity of the surgical tool. Force and position control are both used by the ECU, with these controls performed separately and independently as explained below.

As summarized above, robotic system may be optionally constructed as a master/slave system having a haptic interface (“master”) in communication with the robot (“slave”). The haptic interface is controlled by manual actions of the surgeon during the ocular surgery, with the robot responding to the surgeon's manual inputs to the haptic interface during the ocular surgery via six or more actuators.

In one or more configurations of the latter embodiment, the haptic interface is configured with a force feedback bandwidth of at least 100 hertz (Hz). The scaling logic is operable for selectively reducing a feedback gain for encountered frequencies of less than about 20 Hz and for selectively increasing the feedback gain for frequencies of greater than about 20 Hz. This approach increases tool-tissue interaction feedback while reducing surgeon fatigue.

The robotic system many be equipped with first and second sets of force/torque sensors. The first set of force/torque sensors is operable for sensing input forces/torques imparted by the surgeon to the haptic interface, for outputting input force/torque signals indicated of the measured input forces/torques to the ECU. The second set of force/torque sensors is operable for sensing tool forces/torques imparted by the surgical tool(s) to the eye, and for outputting tool force/torque signals indicative of the measured tool forces/torques for force feedback to the master/haptic interface. The ECU receives the input force/torque signals and the tool force/torque signals and uses the received signals in the overall control of the robot's actions relative to the eye.

At least six linear amplifiers may be disposed between the ECU (or another designated motor controller) and slotless brushless DC (BLDC) motor embodiments of the electric motors of the robot, i.e., one linear amplifier per slotless BLDC. Each linear amplifier reduces interference with the tool force/torque signals, including any encountered electromagnetic interference (EMI) and radio-frequency interference (RFI) with transmitted force/torque sensor and sine-cosine encoder signals as set forth below. Slotless BLDC motors markedly reduce torque ripple relative to slotted BLDCs, and are thus essential for optimal force control and force/tactile feedback within the scope of the disclosure.

The robotic system is operable for sensing a tremor frequency of a hand tremor. Such a hand tremor, typically about 8-15 Hz, e.g., 10-12 Hz, may be imparted by the surgeon to the haptic interface when the surgeon manipulates the haptic interface. Sine cosine encoders present on the haptic interface and used by the haptic device for 6-DOF position control will output a signal indicative of the measured tremor frequency. For clarity, this signal is therefore referred to hereinafter as a tremor signal. The ECU in such an embodiment may filter out the tremor frequency during the ocular surgery. For instance, the ECU may use an adaptive tremor filter to filter out the tremor frequency using a notch filter having an adaptable center frequency, doing so without latency or lag. The adaptable center frequency may be adaptive during the ocular surgery in response to the tremor signal, e.g., using phase-locked loop (PLL) control or similar electronics and algorithms.

The surgical tool may optionally include a pair of surgical tools, in which case the ocular surgery is a bimanual/two-handed process. The ECU in such an implementation may include instability logic operable for preventing instability or oscillations when the pair of tools simultaneously interact with a rigid body during the ocular surgery. For example, the surgical tools may include a pair of forceps configured to grasp an intraocular lens (IOL) or an intraocular foreign body (IOFB), with the IOL or IOFB in this example acting as the rigid body.

An optional single force parameter interface such as a manually operated knob or slider on a surgeon-accessible user interface (UI) may be used for downscaling a feedforward torque/force gain and a feedback torque/force gain. This action simultaneously downscales or limits a tool force imparted to the eye and simultaneously upscales tactile feedback because of the inherent inverse relationship between feedforward and feedback gain. In such an implementation, the force parameter interface forms a single control device operable by the surgeon when downscaling a force/torque to the robot from the haptic interface, as well as when upscaling a force/torque (tactile) feedback to the surgeon via the haptic device.

During the ocular surgery, the ECU may determine whether the surgical tool moves toward or away from the eye, such as when the patient moves in the latter case. The aforementioned scaling logic is operable for reducing velocity of the surgical tool when the surgical tool moves via the robot toward the eye, and for increasing the velocity of the surgical tool when the surgical tools moves away from the eye. This additional feature allows the surgical tool(s) to safely withdraw in response to patient head motion while increasing precision when manipulating tissue.

The electric motors of the robot when constructed as the slotless BLDC motors are operable for positioning and/or actuating the surgical tool(s). Six or more sine-cosine encoders are included in such an embodiment of the slotless BLDC motors, with each of the sine-cosine encoders being operable for measuring or otherwise sensing an angular position for determining rotational position of a respective one of the slotless BLDC motors and performing motor commutation thereof. Among other potential benefits, use of the optional linear amplifiers would help to prevent EMI and RMI from interfering with any sine-cosine encoder and especially force/torque sensor signals.

The above-described features and advantages and other possible features and advantages of the present disclosure will be apparent from the following detailed description of the best modes for carrying out the disclosure when taken in connection with the accompanying drawings.

### BRIEF DESCRIPTION OF THE DRAWINGS

FIG. 1 is an illustration of an ocular surgery performed using a robotic system that is constructed as set forth herein.

FIG. 2 is an illustration of a force control interface usable as part of the robotic system of FIG. 1.

FIG. 3 illustrates a representative notch filter usable for adaptively filtering a hand tremor according to an aspect of the disclosure.

The solutions of the present disclosure may be modified or presented in alternative forms. Representative embodiments are shown by way of example in the drawings and described in detail below. However, inventive aspects of this disclosure are not limited to the disclosed embodiments. Rather, the present disclosure is intended to cover alternatives falling within the scope of the disclosure as defined by the appended claims.

### DETAILED DESCRIPTION

Embodiments of the present disclosure are described herein as examples. Other embodiments can take various and alternative forms. The Figures are not necessarily drawn to scale. Some features may be exaggerated or minimized to show component details. Therefore, specific structural and functional details disclosed herein are not to be interpreted as limiting, but merely as a representative basis for teaching one skilled in the art to variously employ the present disclosure.

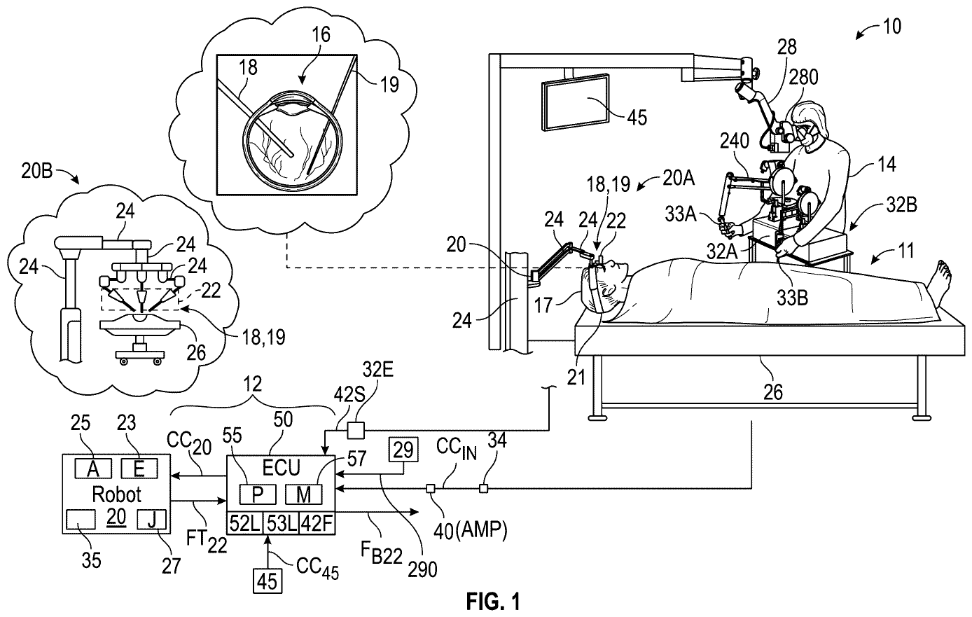

Referring to FIG. 1, an ocular surgery 10 is illustrated within which a robotic system 12 is used to assist a surgeon 14 when performing microsurgery on an eye 16 of a patient 11. Surgeries performed with the assistance of the robotic system 12 may include, e.g., vitreoretinal, cataract, or glaucoma surgeries, or any other surgery in which the surgeon 14 requires access to the eye 16 via one or more surgical tools 18 and/or 19. The robotic system 12 includes an electronic control unit (ECU) 50 and a surgical robot 20, shown in alternative constructions as robots 20A or 20B. The robot 20 may include serial arms/serial kinematics, parallel kinematics, or hybrid parallel-serial kinematics in different embodiments. The robot 20 in turn is communication with the ECU 50, for instance via hardwired transfer conductors and/or wireless pathways, such that the robot 20 is remotely controlled by electronic control signals (CC₂₀) from the ECU 50.

During performance of vitreoretinal, cataract, glaucoma, and other ocular surgeries, a patient may be positioned on an operating platform 26 or on another suitable surgical stretcher, with the surgeon 14 possibly seated on a stool (not shown) close to the patient's head 17, with remote station placement also being possible within the scope of the disclosure. Respective heights of the operating platform 26 and the stool may be adjusted with the assistance of automatic and/or manual knobs, levers, or foot pedals (not shown) in a typical implementation. The surgeon 14 may view magnified images of the eye 16 using an analog or digital ophthalmic microscope 28, e.g., through analog or digital oculars 280 or on a heads-up display using polarized three-dimensional (3D) glasses (not shown) or an auto-stereoscopic display. Although omitted from FIG. 1 for illustrative simplicity, an optical head of the microscope 28 may contain an objective lens and zoom section providing distinct levels of magnifications, a controllable light source, and other hardware and software components.

The ECU 50 of FIG. 1 acts as an electronic controller in the present application, and therefore may employ combinations of Application Specific Integrated Circuit(s) (ASIC), Field-Programmable Gate Array(s) (FPGA), electronic circuit(s), central processing unit(s), e.g., microprocessor(s) and associated non-transitory memory component(s) in the form of digital data storage mediums including memory and storage devices (read only, programmable read only, random access, hard drive, etc.). The ECU 50 therefore includes one or more processors (P) 55 and an application-suitable amount of computer-readable storage medium/memory (M) 57 containing computer-readable/executable instructions for controlling performance of the robot 20 in response to manipulation of the haptic interface(s) 33A, 33B by the surgeon 14.

The non-transitory memory component is capable of storing machine readable instructions in the form of software or firmware programs or routines, combinational logic circuit(s), input/output circuit(s) and devices, signal conditioning, buffer circuitry and other components, which can be accessed by and executed by one or more processors to provide the described functionality. Communication in the course of controlling the robot 20 includes exchanging data signals, including, for example, electrical signals via a conductive medium, electromagnetic signals via air, optical signals via optical waveguides, etc. Data signals may include discrete, analog and/or digitized analog signals representing inputs from sensors, actuator commands, and communication between controllers.

The robot 20 is illustrated according to two possible implementations, i.e., robots 20A and 20B. The robot 20A may be constructed as a head-attachable device in which the robot 20A is counterbalanced and securely strapped to the head 17 of the patient 11, e.g., via straps 21. An end-effector 22 of the robot 20A is connected to and supports one or more surgical tools 18 and/or 19. The end-effector 22 in turn is coupled to six (or more) links 24 interconnected by revolute joints (J) 27, which in turn may be actuated/active joints, e.g., via a corresponding electric motor 25 driven by electronic control signals (CC₂₀) from the ECU 50. Examples of such a robot 20A are disclosed in U.S. patent application Ser. No. 17/151,829, which issued on Nov. 7, 2023, as U.S. Pat. No. 11,806,105B2, and U.S. patent application Ser. No. 17/649,569, which published on Aug. 11, 2022, as U.S. Patent Application Publication No. US2022/0249183A1, both of which are hereby incorporated in their respective entireties for all purposes.

Alternatively, the robot 20B having the end-effector(s) 22 may be securely mounted, for instance to the operating platform 26 on which the patient 11 rests during the ocular surgery 10. As with the robot 20A, the robot 20B includes six or more links 24 and six or more electric motors/rotary actuators (A) 25 connected to the links and controllable via the ECU 50. The electric motors 25 of robots 20A or 20B may be configured as slotless brushless direct current (BLDC) motors operable for positioning and/or actuating the surgical tools 18 and/or 19. The robots 20A and 20B also include one or more sine-cosine position encoders (E) 23 operable for measuring/sensing an angular position of the slotless BLDC motors and for performing motor commutation thereof. Use of slotless BLDCs herein is intended to minimize torque ripple in both master-slave (robot 20A) and autonomous (robot 20B) implementations.

Regardless of its construction, the robotic system 12 senses and reports a set of parameters 290, for instance via a smart connector 29. As noted above, the smart connector 29 is disposed between the surgical tool(s) 18 and/or 19 and the end-effector 22. The parameters 290 as envisioned herein may include a tool mass and moments of the surgical tools 18 and 19, the construction of which will vary depending on the ocular surgery 10. The parameters communicated by the smart connector 29 also include moments, lengths, and offsets (if non-axial), all of which are communicated to the ECU 50. A representative construction of the smart connector 29 is disclosed in U.S. patent application Ser. No. 18/796,670, which was filed on Aug. 7, 2024, and which is hereby incorporated by reference in its entirety for all purposes. The ECU 50 can thereafter control states of the various electric motors 25 and ensure that the robot 20 is effectively gravity compensated in all of its available poses. This feature enhances dexterity, reduces fatigue and increases safety.

Counter-balancing is used herein to provide additional benefits. Counter-balancing supports the entire base of the robot 20. The base may employ a counterbalance mount such as but not limited to a counterweight or one or more gas springs. Exemplary approaches for implementing counterbalanced mounts are disclosed in U.S. patent application Ser. No. 18/592,640, which published on Sep. 5, 2024, as U.S. Patent Application Publication No. 2024/0293192A1, which is hereby incorporated by reference in its entirety for all purposes.

In the representative robotic system 12 illustrated in FIG. 1, the robot 20 and the surgical tool(s) 18 and/or 19 are configured, in response to electronic control signals CC₂₀ from the ECU 50, to perform a surgical task on the eye 16 during the ocular surgery 10. As part of this effort, the ECU 50 employs scaling logic 52L to downscale or limit a tool force imparted to the eye 16 by the surgical tool(s) 18, 19. Separately, the scaling logic 52L also scales or limits a tool velocity of the surgical tool(s) 18, 19 when the surgical tools move toward (downscales) or away (upscales) from the eye 16.

For instance, the ECU 50 may determine, during the course of the ocular surgery 10, whether the surgical tool(s) 18 and/or 19 move toward or away from the eye 16. The ECU 50 may do so using the sine-cosine position encoders 23 on the robot 20 and sensed data from one or more cameras or other sensors (not shown), knowledge of position commands from the ECU 50 based on actions of the surgeon 14 via the haptic interface 33A, 33B, etc. Using the scaling logic 52L, the ECU 50 is operable for selectively reducing a velocity of the tool(s) 18 and/or 19 when the surgical tool(s) 18 and/or 19 move toward the eye 16 (“toward” velocity downscaling) and for increasing the velocity to normal of the surgical tool(s) 18 and/or 19 when the surgical tools 18 and/or 19 move away from the eye 16 (“away” velocity upscaling). The robotic system 12 is configured to address this and various other problems of types commonly associated with robot-assisted ocular surgery 10. Other possible input signals to the ECU 50 include output signals (CC₄₅) from an optional force parameter interface 45.

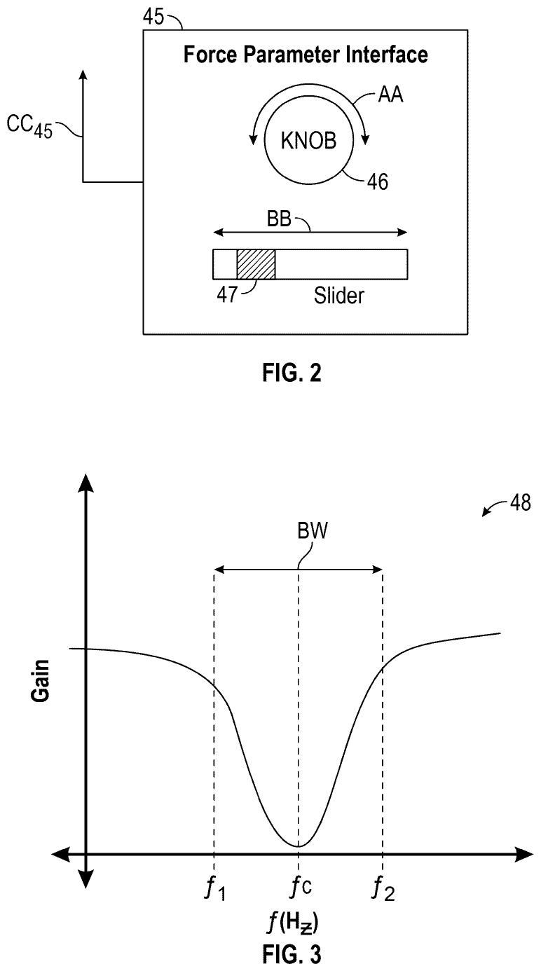

Referring briefly to FIG. 2, a user interface (UI) 44 in the operating suite of FIG. 1 may be used as part of the robotic system 12 of FIG. 1, and may include the force parameter interface 45 noted above. The force parameters interface 45 may be embodied as a manually operated knob 46 or slider 47. While both the knob 46 and the slider 47 are illustrated for simplicity, an actual implementation may include either the knob 46 or the slider 47.

When the surgeon 14 of FIG. 1 manipulates the force parameter interface 45, such as by rotating the knob 46 (arrow AA) or translating the slider 47 (arrow BB) to a desired set point, the ECU 50, responding to the output signals (CC₄₅), downscales a feedforward torque/force gain and upscales a feedback torque/force gain value. This action in turn downscales or limits a tool force imparted to the eye 16 of FIG. 1 by the surgical tool(s) 18 and/or 19 of FIG. 1. The force control interface 45 thus forms a single control device operable for downscaling a force/torque to the robot 20 from the haptic interface 33A, 33B of FIG. 1, and upscaling a force/torque feedback to the haptic interface 33A, 33B, as needed at the discretion of the surgeon 14.

Force scaling inversely to force feedback gain, i.e., force “zoom” control, helps optimize safety and precision relative to using position control alone. Force control gain as a parameter setting reduces tool forces in 6-DOF relative to the 6-DOF forces/torques applied to the haptic interface 33A, 33B by the surgeon 14. 6-DOF force/torque feedback in turn is scaled in the opposite direction, i.e., upscaled if the applied force is downscaled, and vice versa. The present hybrid position and force control approach is thus distinguishable from typical position-only robotic control.

Referring once again to FIG. 1, the surgical tools 18 and/or 19 may include a pair of the surgical tools 18 and 19. The surgical tool 18 and the surgical tool 19 may be operated simultaneously such that the ocular surgery 10 is a two-handed/bimanual process. The ECU 50 in such a use scenario may also include instability logic 53L operable for preventing instability or oscillations when the pair of surgical tools 18 and 19 simultaneously interact with a rigid body during the ocular surgery 10, e.g., with an intraocular lens (IOL) or an intraocular foreign body (IOFB). For example, the pair of surgical tools 18 and 19 may include a pair of pars plana forceps configured to grasp the IOL or IOFB as such a rigid body.

During a representative cataract surgery, the surgeon 14 may be required to manually track and compensate for movement of the eye 16. It is recognized herein that automated pupil/limbus eye tracking techniques during ocular surgery remain imprecise, have insufficient degrees of freedom, especially when attempted in a six degree of freedom (6-DOF) robot-assisted surgical operating environment of the type considered herein. For several reasons, effective real-time eye tracking during ocular surgery is only feasible using multi-sensor data fusion. 4-DOF remote center of compliance kinematics are insufficient, with 6-DOF being required (with no redundant or additional DOF). However, inverse kinematics calculation for a 6-DOF serial robot application, i.e., roll, pitch, yaw, and Y, Y, and Z axis translation, requires approximation through the iterative solving of a 16th order polynomial expression. At present, viable high-resolution, non-ambiguous, real-time 6-DOF head or eye position/pose sensing technologies remain unavailable. Moreover, artificial intelligence (AI)-based models are not practicable for use in predicting sudden, rapid, complex head and eye movements common during surgery under topical or local anesthesia.

Accordingly, the robotic system 12 of FIG. 1 is constructed to be highly robust to movements of the head 17 and resulting motion of the eye 16 of the patient 11 absent the use of such tracking techniques. Head movement, which is not constrained during cataract surgery, is only minimally constrained during macular surgery. Motion of the patient's head 17 or eye 16 can result from a myriad of factors, including sudden awakening from sedation or an episode of sleep apnea, or from the patient's startle reflex due to sudden/loud noises. Other factors include head tremors, involuntary myoclonic jerks, hiccups, restless leg syndrome, movement disorders such as Parkinson's, stroke, or restless leg syndrome. Additionally, topical and intracameral anesthesia do not result in any paralysis of extraocular muscles (akinesia) during surgery. As a result, unpredictable movements of the eye 16 with large amplitudes may occur. Features of the robotic system 12 therefore account for such movement, with the robot 20A in particular being useful for eliminating relative motion between the robot 20 and the patient when the patient moves their head 17.

Other factors complicating robot-assisted ocular surgeries include, e.g., floppy iris, zonular dehiscence, wound leak, shallow chamber, and possible capsule tears or ruptures. Moreover, lens material, the vitreous of the eye 16, and a detached retina are all highly mobile with unpredictable motion. Debulking of lens material, vitreous, scar tissue continuously alters geometry, changes biomechanical properties, and decreases viscosity. The lens material, cornea, capsule, and vitreous are all relatively transparent, which is a significant challenge for stereo imaging or optical coherence tomography (OCT). The lens material also produces reflective effects, obscuration issues, obliquity issues, and projection artifacts, all of which further complicate the surgical process. With respect to OCT used alone, image ambiguity ensures that reliable, safe, robust image guidance is not possible with the present state of the art, even with AI assistance.

Repurposed industrial robots or same sized arm type purpose-built robots also have various significant limitations when applied in the context of ocular surgery. Such robots tend to have excessive mass/inertia which results in decreased positioning accuracy, over-shoot, static friction (“stiction”), and other drawbacks, which can increase risk of ocular damage in the event of a control malfunction. Increased mass/inertia also decreases the ability of the surgeon to sense small tissue forces, markedly reducing bandwidth of force control and force feedback. When using serial kinematics as noted above, there may be a tolerance and/or friction stack up, either or both of which can further complicate the operating effectiveness of the robot 20. The robotic system 12 of FIG. 1 is therefore configured to address the various concerns noted above, with other technical features such as direct drive, passively back-drivable motors and combined position and force control law, 20-100 Hz force feedback, and 6-DOF force sensing at the end-effector of the robot 20 in lieu of motor-based current sensing.

The robotic system 12 of FIG. 1 may be fully autonomous in a possible implementation. For autonomous surgeries, the ECU 50 may control motion of the robot 20 via the electronic control signals (CC₂₀), for instance using model-based and/or AI or machine learning (ML)-based techniques. Such approaches may be assisted by real-time sensing, computer vision, neural networks, and programmed task sequences.

Alternatively, the robotic system 12 may be implemented as a master/slave construction of the robotic system 12, e.g., using the robot 20A. In such an embodiment, the robotic system 12 may include (i) at least one low-friction low inertia haptic device 32A, 32B having a corresponding 6-DOF haptic interface 33A, 33B, and (ii) a subordinate secondary device, in this case the robot 20 in the form of robot 20A. The haptic device 32A, 32B is controlled by the surgeon 14 via manual inputs to the haptic interface 33A, 33B during the ocular surgery. In an actual implementation, the surgeon 14 may often manipulate two different surgical tools, and therefore the robotic system 12 may employ dual haptic devices 32A, 32B and dual haptic interfaces 33A and 33B, i.e., one per surgical tool 18 and 19. The robot 20A responds to the surgeon's manual inputs to the haptic interface 33A, 33B. Depending on the construction of the haptic device 32A, 32B, the haptic interface 33A, 33B may include handles or another graspable device that the surgeon 14 can hold and manipulate while viewing magnified images of the eye 16, e.g., through the oculars 280 of the microscope 28.

Still referring to FIG. 1, the haptic device 32A, 32B used herein may be embodied as a 6-DOF force feedback-based device. Various commercially available options exist for the haptic device 32A, 32B, for instance the Phantom® Omni or the Phantom® Premium™ devices, both of which are commercially available through Geomagic®. When constructed for use as part of the robotic system 12 of FIG. 1, 6-DOF models may be used to provide three translational and three rotational DOF in output capabilities to mimic full range of motion of a hand and wrist of the surgeon 14.

The haptic device 32A, 32B may include six or more back-drivable rotary or linear motors (no gears or harmonic drives) and corresponding rotary or linear encoders, as appreciated in the art, and a set of force/torque sensors 34, which may be used to generate the force/torque input signals (CC_{IN}). As appreciated in the art, manipulation of the 6-DOF haptic interface 33A, 33B drives six motors of the haptic device 32A, 32B, causing six encoders to read distinct positions of (six) corresponding links 240 of the haptic device 32A, 32B. Simultaneously, a 6-DOF force/torque sensor of the haptic device 32A, 32B senses movement of the links 240 as they impart forces on a structure of the haptic device 32A, 32B. Collectively, the haptic device 32A, 32B communicates its signal outputs to the ECU 50 for processing.

Continuing with the discussion of haptic sensing, the first set of force/torque sensors 34 may be positioned at or near a kinematic junction of a handle of the haptic interface 33A, 33B, i.e., where the above-mentioned handle meets the lightweight, 6-DOF haptic device 32A, 32B with slotless BLDC motors for feedback and encoders for position input, as discussed above. The force/torque sensors 34 are operable for measuring or otherwise sensing input forces/torques imparted by the surgeon 14 to the haptic interface 33A, 33B when the haptic interface 33A, 33B is manipulated or acted on by the surgeon 14. The force/torque sensors 34 are also operable for outputting the force/torque input signals (CC_{IN}) to the ECU 50, with the force/torque input signals (CC_{IN}) being indicative of the measured input forces/torques. The ECU 50 then transmits electronic control signals (CC₂₀) to the robot 20 to cause the robot 20, e.g., 20A, to perform surgical tasks on the eye 16.

Ideally, the robot 20A is controlled via hybrid position-force control, so that the 6-DOF position/orientation and 6-DOF force/torque real time data processed by the ECU 50 controls robot position and applied force in 6-DOF. As appreciated in the art, there is a key difference between control law and parameter settings. Hybrid force control may be used herein in lieu of conventional position (i.e., position only) control, as hybrid force control requires passive backdrivability, slotless motors, and force/torque sensing, along with low friction, low inertia, and no gear or harmonic drives. In contrast, parameter setting refers to force control gain or limits on the UI 46 (see FIG. 2).

The ECU 50 of FIG. 1 may also transmit force feedback signals (FB₂₂) to the haptic device 32A, 32B to allow the surgeon 14 to perceive a simulated feel of contact between the surgical tool(s) 18 and/or 19 and tissue of the eye 16. Similarly, a second set of force/torque sensors 35 is disposed on the robot 20 and operable for measuring/sensing actual tool forces/torques imparted by the surgical tool(s) 18 and/or 19 to the eye 16, and for outputting tool force/torque signals (FT₂₂) indicative of the measured tool forces/torque. Therefore, feedback and feedforward values are used herein to closely coordinate the actions of the robot 20 with manual inputs to the haptic device 32A, 32B by the surgeon 14. The haptic device 32A, 32B may have a force feedback bandwidth of at least 100 Hz in one or more embodiments. In this case, the scaling logic 52L is configured to selectively reduce a feedback gain for frequencies of less than about 20 Hz, and to selectively increase the feedback gain for frequencies greater than about 20 Hz.

In one or more embodiments, a linear amplifier (AMP) 40 may be disposed between the ECU 50 and each of six (or more) electric motor(s) 25 of the robot 20. Such linear amplifiers 40 are operable for reducing interference with the tool force/torque signals (FT₂₂) indicative of the measured tool forces/torques as noted above. Relative to pulse width modulation (PWM) amplifiers commonly employed in medical motor drives to conserve packaging space and reduce power consumption and cost, the linear amplifiers 40 used herein are intended to eliminate amplitude EMI spikes that would otherwise be inductively coupled to low signal amplitude cables connecting 6-DOF force/torque sensors to the ECU 50. Force/torque sensors used as part of the robot 20 would be located where the surgical tool(s) 18 and/or 19 connect to the end-effectors 22 of FIG. 1. The use of linear amplifiers 40 avoids this issue.

The haptic device 32A, 32B includes tremor sensing functionality by virtue of the use and presence of 6-DOF sine-cosine encoders 32E of the haptic device 32A, 32B. That is, 6-DOF encoders 32E on the haptic device 32A, 32B sense its position. Thus, the encoders may be used to sense tremors and report the sensed tremor frequency as a tremor signal 42S. Using sensed position and the first and second derivates, therefore, the haptic device 32A, 32B is operable for (i) sensing a tremor frequency of a hand tremor that is imparted by the surgeon 14 to the haptic interface 33A, 33B, and (ii) outputting the tremor signal 42S indicative of the measured tremor frequency.

The ECU 50 in such an embodiment is configured to filter out the tremor frequency when hand tremors occur during the ocular surgery 10 of FIG. 1. Tremor frequency filtering may occur using an adaptive tremor filter 42F, with disclosed approaches for adaptively filtering sensed hand tremors are configured to avoid phase lag (latency). Such an approach is distinguishable from existing low pass filter (LPF)-based tremor filtering at 5 Hz, as will be appreciated by those of ordinary skill in the art.

Referring to FIG. 3, the adaptive tremor filter 42F noted above with reference to FIG. 1, in accordance with an embodiment, may use a notch filter 48 for this purpose. The notch filter 48 may have an adaptive bandwidth (BW) or an adaptive center frequency range (f₁-f₂). To this end, the adaptive center frequency f_{c} of the notch filter 48 is adaptive, i.e., modifiable, in real-time during the ocular surgery in response to the tremor signal 42S of FIG. 1 in this example embodiment. As a possible approach, the ECU 50 of FIG. 1 may implement a phase-locked loop (PLL). As appreciated in the art, use of a PLL allows generation of an output signal having a phase that is related to a phase of an input signal. A potential benefit of applying a PLL to the adaptive tremor filter 42F is the capability of quickly tracking frequency changes in the input signal, in this case the tremor signal 42S (FIG. 1). Another advantage of a phase-locked control loop is that the tremor may vary in frequency and may be in volitional movement range.

The robotic system 12 of FIG. 1 as described above therefore provides the requisite structure and function for safely performing ocular surgery with the assistance of high-DOF robots such as the robot 20 of FIG. 1. While autonomous ocular surgeries remain possible with the present teachings, the present teachings may be of particular advantage when applied to surgeries performed with the assistance of the 6-DOF haptic device 32A, 32B, i.e., with the surgeon's force and position input and resulting commands being manually provided via interaction with the haptic interface 33A, 33B. The present teachings also overcome ambiguity and geometric uncertainty associated with imaging/OCT-driven robot-assisted surgeries.

As those of ordinary skill in the art will understand, various features illustrated and described with reference to any one of the Figures can be combined with features illustrated in one or more other Figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Unless specifically disclaimed, use of the singular herein includes the plural and vice versa, e.g., indefinite articles “a” and “an” should generally be construed as meaning “one or more”. Likewise, the words “and” and “or” shall be both conjunctive and disjunctive, the words “any” and “all” shall both mean “any and all”, and the words “including,” “containing,” “comprising,” “having,” and the like shall each mean “including without limitation.” Moreover, words of approximation, such as “about,” “almost,” “substantially,” “generally,” “approximately,” and the like, may each be used herein to denote “at, near, or nearly at,” or “within 0-5% of,” or “within acceptable manufacturing tolerances,” or any logical combination thereof, for example.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

What is claimed is:

1. A robotic system for performing an ocular surgery, comprising:
an electronic control unit (ECU);
a robot in communication with the ECU, the robot having an end-effector, six or more links, and six or more electric motors each operable for moving one or more links and controllable via the ECU, wherein the robot is configured with an all-pose gravity compensation construction; and
a surgical tool connected to the end-effector, wherein:
the robot and the surgical tool, in response to electronic control signals from the ECU, are configured to perform a surgical task on an eye during the ocular surgery;
the ECU includes scaling logic operable for downscaling or limiting a tool force imparted to the eye by the surgical tool and/or a tool velocity of the surgical tool; and
the all-pose gravity compensation construction is configured to minimize a perceived weight of the surgical tool during the ocular surgery.

2. The robotic system of claim 1, wherein the robotic system is fully autonomous.

3. The robotic system of claim 1, wherein the robotic system is a master/haptic-slave system comprising: (i) a haptic device including a haptic interface, and (ii) the robot, wherein the haptic device is manipulated by a surgeon via a manual input to the haptic interface during the ocular surgery, and the robot is remotely controlled by electronic control signals from the ECU in response to the manual input.

4. The robotic system of claim 3, wherein the haptic device has a force feedback bandwidth of at least 100 hertz (Hz), the scaling logic is configured to selectively reduce a feedback gain for frequencies of less than about 20 Hz, and the scaling logic is configured to selectively increase the feedback gain for frequencies greater than about 20 Hz.

5. The robotic system of claim 3, further comprising:
a first set of force/torque sensors operable for measuring input forces/torques imparted by the surgeon to the haptic interface when the haptic device is manipulated by the surgeon, and for outputting input force/torque signals indicated of the measured input forces/torques; and
a second set of force/torque sensors operable for measuring tool forces/torques imparted by the surgical tool to the eye, and for outputting tool force/torque signals indicated of the measured tool forces/torques.

6. The robotic system of claim 5, further comprising:
at least six linear amplifiers each disposed between the ECU and a respective one the one or more electric motors, the at least six linear amplifiers being operable for reducing interference with the tool force/torque signals.

7. The robotic system of claim 5, wherein the haptic device includes sine-cosine encoders operable for sensing a tremor frequency of a hand tremor that is imparted by the surgeon to the haptic interface when the haptic interface is manipulated by the surgeon, and for outputting a tremor signal indicative of the tremor frequency, the ECU being configured to filter out the tremor frequency during the ocular surgery.

8. The robotic system of claim 7, wherein the ECU includes an adaptive tremor filter operable for filtering out the tremor frequency using a notch filter having an adaptive center frequency, the adaptive center frequency being adaptable by the ECU during the ocular surgery in response to the tremor signal using a phase-locked control loop.

9. The robotic system of claim 5, wherein the surgical tool includes a pair of surgical tools such that the ocular surgery is a bimanual process, and wherein the ECU includes instability logic operable for preventing instability or oscillations when the pair of tools simultaneously interact with a rigid body during the ocular surgery.

10. The robotic system of claim 9, wherein the pair of tools includes a pair of pars plana forceps configured to grasp an intraocular lens as the rigid body.

11. The robotic system of claim 3, further comprising:
a force parameter interface configured for downscaling a feedforward torque/force gain and a feedback torque/force gain to thereby downscale or limit a tool force imparted to the eye, such that the force parameter interface forms a single control device operable for downscaling a force/torque to the robot from the haptic interface and upscaling a force/torque feedback to the haptic device.

12. The robotic system of claim 11, wherein the force parameter interface is located on a user interface, and includes a manually operated knob or slider on the user interface.

13. The robotic system of claim 1, wherein the ECU is configured to determine, during the ocular surgery, whether the surgical tool moves toward or away from the eye, and wherein the scaling logic is operable for downscaling or reducing a velocity of the tool when the surgical tool moves toward the eye and upscaling or increasing the velocity of the tool when the surgical tools moves away from the eye.

14. The robotic system of claim 1, wherein the robot includes serial kinematics, parallel kinematics, or hybrid parallel-serial kinematics.

15. The robotic system of claim 1, wherein the six or more electric motors are configured as slotless brushless direct current (BLDC) motors operable for positioning and/or actuating the surgical tool, further comprising:
six or more sine-cosine encoders operable for measuring an angular position of the one or more slotless BLDC motors and performing motor commutation thereof.

16. A robotic system for performing cataract surgery, comprising:
an electronic control unit (ECU) having scaling logic, instability logic, and an adaptive tremor filter operable for filtering out a tremor frequency during the cataract surgery using a notch filter, the notch filter having an adaptive center frequency that is adaptable during the cataract surgery in response to a tremor signal; and
a master-slave robotic system, including:
a pair of haptic devices in communication with the ECU;
a robot having at least one end-effector, six or more links, and six or more slotless brushless direct current (BLDC) motors connected to the links and controllable via the ECU, wherein the robot is configured with an all-pose gravity compensation construction; and
a pair of surgical tools each connected to a respective one of the end-effectors via a smart connector, wherein:
the robot, in response to electronic control signals from the ECU, is configured to perform a surgical task on an eye during the cataract surgery;
the ECU is operable, via the scaling logic, for downscaling or limiting a tool force imparted to the eye by the surgical tool and/or a tool velocity of the surgical tool; and
the ECU is operable, via the instability logic, for preventing instability or oscillations when the pair of surgical tools simultaneously interact with an intraocular lens (IOL) or an intraocular foreign body (IOFB) during the cataract surgery.
